(19) United States
(12) Patent Application Publication
(10) Pub. No.: US 2010/0056978 A1
(43) Pub. Date: Mar. 4, 2010

(54) Externally adjustable blood flow valve

(76) Inventors: Lindsay Machan, Vancouver (CA); Daniel Gelbart, Vancouver (CA)

Correspondence Address: DANIEL GELBART, 4706 DRUMMOND DR., VANCOUVER, BC, V6T-1B4, CA

(21) Appl. No.: 12/230,267

(22) Filed: Aug. 27, 2008

(51) Int. Cl. A61M 1/00

(52) U.S. Cl. 604/9

(57) ABSTRACT

A valve installed in a blood vessel can be externally adjusted over a wide range of flow rates. Such a valve can be incorporated in an AV shunt or AV fistula to allow large flow during dialysis and small flow at all other times. The valve can be activated by finger pressure, hypodermic needle or by an electromagnetic field. The valve is shaped like a Venturi tube with round cross section and smooth diameter transition to minimize blood damage and clotting. Hemodynamic properties are further enhanced by keeping the part coming in contact with the blood a single continuous tubing.

### FIELD OF THE INVENTION

The invention is in the medical field and in particular in the field of Arteriovenous Shunts and Fistulas, also known as AV shunts and AV fistulas, used in dialysis.

### BACKGROUND OF THE INVENTION

A significant number of patients suffering from kidney failure require periodic dialysis. In order to speed up the pumping of the blood through the dialysis machine, a fistula is formed, typically in the arm, by connecting the main artery to the vein. The connection can be by cutting the two walls and suturing them together or by using a short graft to connect them. The graft can be made from a blood vessel taken from the patient or made from an artificial material such as Gortex (PTFE fabric), polyurethane or silicone rubber. Such a fistula or graft allows the repeated piercing by a hypodermic needle required by the dialysis process. During dialysis it is desired to have a large blood flow from the artery to the vein but in the period between dialysis sessions such a large flow damages the veins, as the operate at a much lower blood pressure. The process of deterioration of the fistula and vein is known as “intimal hyperplasia”. Current shunt size is a compromise between minimizing this damage and maximizing the flow. It is desired to have an adjustable valve as part of the shunt or connection. The valve can be opened for a large flow during dialysis but the flow can be reduced significantly, down to zero if desired, between dialysis sessions. Prior art valves suggested for AV shunts had a sudden change in cross section and also had pockets of very low blood flow, causing clotting. Other prior art is based on squeezing a tube to reduce flow. When a tube is squeezed the cross section does not stay round, causing spots with low blood flow which generate blood clots. For example, U.S. Pat. No. 7,128,750 proposes many ways of deforming a round tube to reduce flow (shown in FIG. 11), however all of them will increase blood clotting as they have areas with very low flow or no flow. It is desired to have a valve creating as little blood damage and blood clotting as possible. It is known that to minimize blood damage and clotting it is desired to avoid turbulent flow, avoid blind sections and avoid abrupt changes in cross section or wall structure. The most desirable shape is a round tube slowly changing its diameter from large to small and back to large, similar to a Venturi tube. It is an object of the invention to create such a valve. Further objects and advantages of the invention will become apparent by reading the disclosure in conjunction with the drawings.

### SUMMARY OF THE INVENTION

A valve installed in a blood vessel can be externally adjusted over a wide range of flow rates. Such a valve can be incorporated in an AV shunt or AV fistula to allow large flow during dialysis and small flow at all other times. The valve can be activated by finger pressure, hypodermic needle or by an electromagnetic field. The valve is shaped like a Venturi tube with round cross section and smooth diameter transition to minimize blood damage and clotting. Hemodynamic properties are further enhanced by keeping the part coming in contact with the blood a single continuous tubing.

### BRIEF DESCRIPTION OF THE DRAWINGS

FIG. 1 is a general view of an AV shunt placed inside the arm and incorporating a valve.

FIG. 2 is a cut-away isometric view of a valve being operated mechanically by sliding a ring.

FIG. 3A is a longitudinal cross section of a mechanically operated valve in the minimal flow position.

FIG. 3B is a longitudinal cross section of a mechanically operated valve in the maximal flow position.

FIG. 4 is a longitudinal cross section of a hydraulically operated valve in the minimal flow position.

FIG. 5 is an isometric view of a flexible cage used in some of the valve designs.

FIG. 6A is a longitudinal cross section of an electromagnetically operated valve in the minimal flow position.

FIG. 6B is a longitudinal cross section of an electromagnetically operated valve in the maximal flow position.

FIG. 7 is a schematic view of the device for electromagnetic activation of the valve.

### DETAILED DISCLOSURE

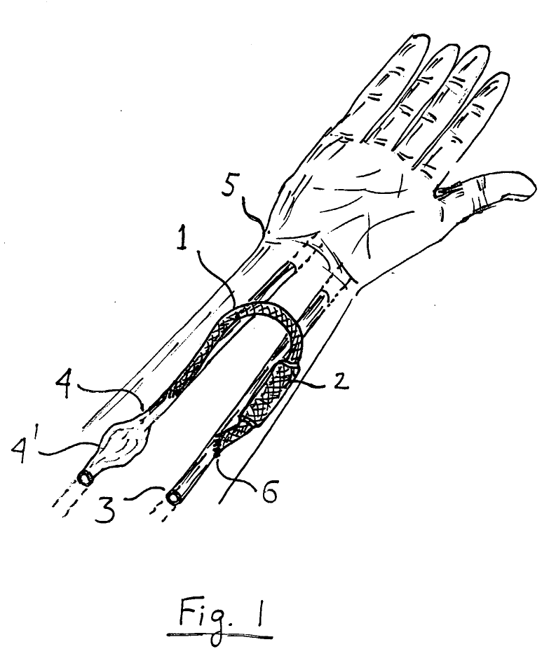

Referring to FIG. 1, an AV shunt 1 connects artery 3 to vein 4 in arm 5 via valve 6. Prior art AV shunts do not include valve 2. During dialysis hypodermic needles are placed in shunt 1 or fistula 4′ formed in the vein. The shunt 1 can be replaced by a direct connection between artery 3 and vein 4. In such a case the valve 2 is installed in the connection. The art of Av fistulas and shunts is well known and need not be further detailed. Valve 2 allows full blood flow during dialysis and restricts blood flow at all other times. The valve can be controlled is several ways, some of the preferred ways are listed here:
A. Direct mechanical control, by sliding or moving part of the valve that can be felt from outside of the body. For example, when installed in the arm, a ring can be caused to slide inside the valve by pushing the tissue above it.
B. Hydraulic control. Injecting or removing a fluid from a section of the valve by using a hypodermic needle controls the flow.
C. Electromagnetic control. Applying a strong magnetic pulse to the area the valve is located can cause a part to move inside the valve, affecting flow. Reversing the effect is done by applying an opposite magnetic field of applying a magnetic field at a different position.
D. Electromagnetic control by heating. Applying an alternating magnetic field can generate heat in a closed circuit electrical path inside the valve. The heat can be used to activate one of many well known heat to mechanical motion converters such as bimetallic elements

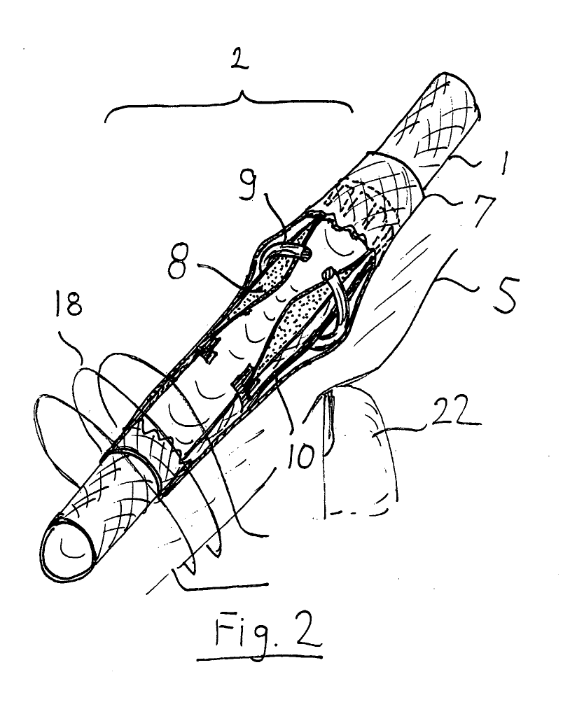

Referring now to FIG. 2 a cut-away view of valve 2 is shown. The valve is made from two layers of flexible tubing: the inner tube 1, which forms the shunt or anastomosis between blood vessels, and an outer tube 7 sealed at its ends to tube 1. The gap between the tubes contains the valve mechanism. A flexible Venturi shaped body 8 is constrained to have a small opening by ring 9. When ring 9 is moved to the end of body 8, it expands elastically and provides full flow. Intermediate flows can be achieved by placing ring 9 at intermediate positions. Sometimes it is advantageous to add a flexible metal cage 10 to body 8. A clearer view of metal cage 10 is shown in FIG. 5. The space between tube 1 and tube 7 is filled with a saline solution. Because the inside of tube 7 stays continuous and is not interrupted by steps, leaflets, vanes or similar objects the amount of turbulence is minimized. The valve can be sized to keep the Reynolds number of the flow below Re=3000 to avoid turbulence. Avoiding turbulence minimized damage to red blood cells. The smooth Venturi (or nozzle) shape avoids pockets where blood is not flowing. Such low flow points cause blood clotting. This is the reason why it is preferred not to shut off the flow completely; a small flow will prevent blood clots. Inner tube 1 can be an AV shunt but can also be an existing vein or artery, as well as a piece of vein an artery added to the body (from human, animal or artificial source). When tube 1 is a blood vessel the ends of tube 7 need to be connected to it by suturing, medical adhesive or any other sealing method. This is done to prevent micro-organisms and tissue to grow inside the valve. Tubes 1 and 7 do not need to be hermetically sealed, as long as no living cells can penetrate the space between them. The ability of saline molecules to penetrate is not a problem and actually can be an advantage.

Ring 9 forms a bulge on tube 7 that can be felt from the outside. By applying finger pressure from outside the body, as shown by finger 22 applying pressure to arm 5, the ring can be moved to adjust blood flow. If ring 9 is made of metal it can also be moved by the application of a magnetic field. A coil 18 wrapped around arm 5 can be used to attract ring 9 when ring is made of a ferromagnetic material such as type 400 stainless steel and can also be used to repel ring 9 if made from a good electrical conductor such as aluminum, silver or copper. By moving coil 18, ring 9 can be pushed or pulled in the desired direction. FIG. 3A shows a cross section of the valve in the minimal flow position and FIG. 3B is a cross section in the maximal flow position. Detents 11 can be added for a positive lock at the desired positions.

By the way of example, tubes 1 and 7 can be made of Gortex (woven PTFE), Dacron, polyurethane, silicone rubber or many other flexible materials. They can be treated with beneficial surface coatings such as drug eluting coating, hydrophobic coating etc. Flexible body 8 can be made from a silicone or polyurethane rubber, either as a solid or a closed cell foam. Optional cage 10 (shown in FIGS. 2 and 5) can be made of a polymer or a flexible metal such as spring tempered stainless steel. Typical thickness for the cage wall is 0.1-0.3 mm. Since tubes 1 and 7 have some porosity, the pressure of saline solution 12 will come to equilibrium with the outside pressure.

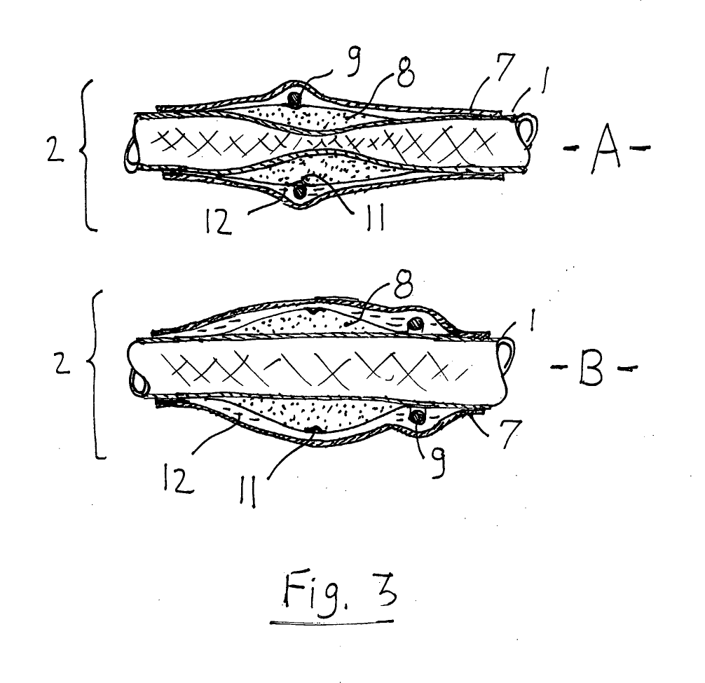

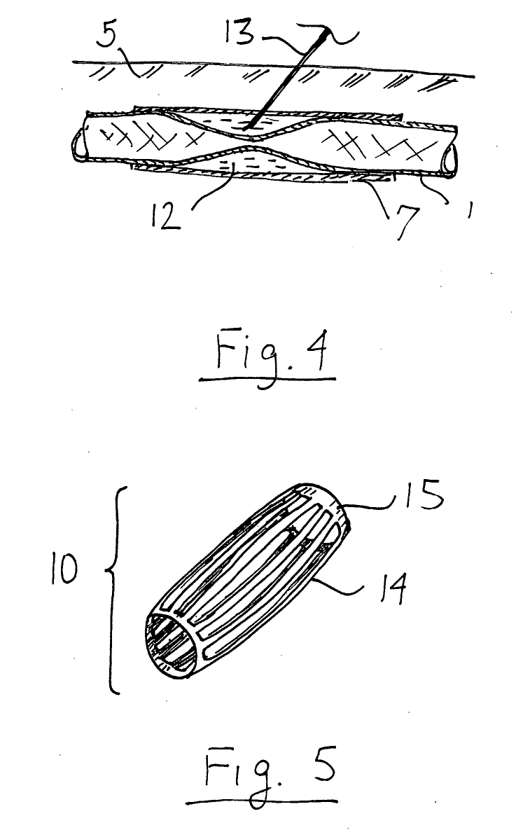

A simpler valve, activated hydraulically by a hypodermic needle, is shown in FIG. 4. Tube 7 is made more rigid than inner tube 1. When the amount of saline solution 12 in the space between tubes is changed, the shape of the inside Venturi-like passage changes. Solution 12 is accessed by a hypodermic needle 13 during dialysis. An alternate hydraulic activation, not requiring piercing the skin, can be done by connecting solution 12 to a small fluid reservoir via a miniature pump, similar to the practice with urethral valves and penile implants. The pump is activated by pressing on the outside of the body at a known point and the pressure is released by pressing or twisting a different point. The reservoir can be quite small, in the order of 1-2 c.c.

FIG. 5 is an isometric view of the flexible cage used in FIGS. 2, FIG. 6 and FIG. 7. Cage 10 is made of flexible ribs 14 held together by rings 15. Cage 10 can be made of metal or polymeric material. It can be used to add strength and flexibility to the valve and force the valve to deform in a desired manner when activated.

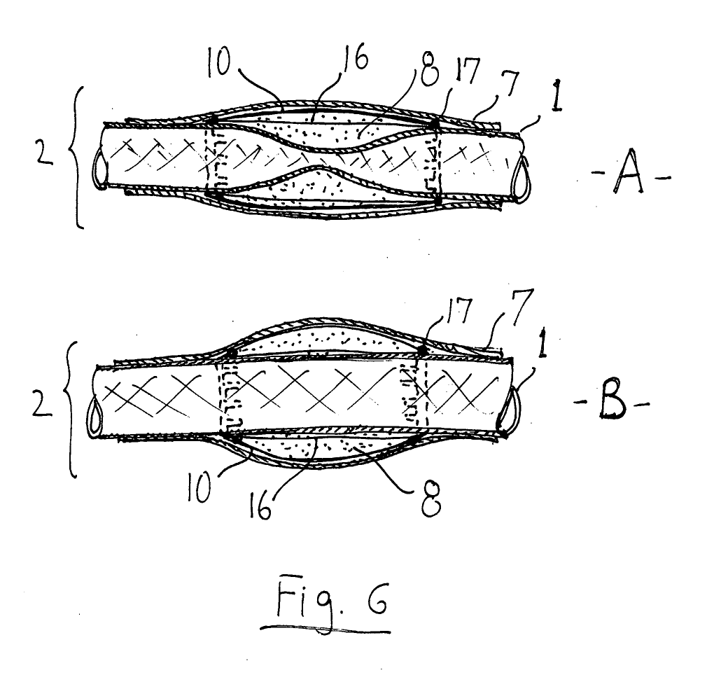

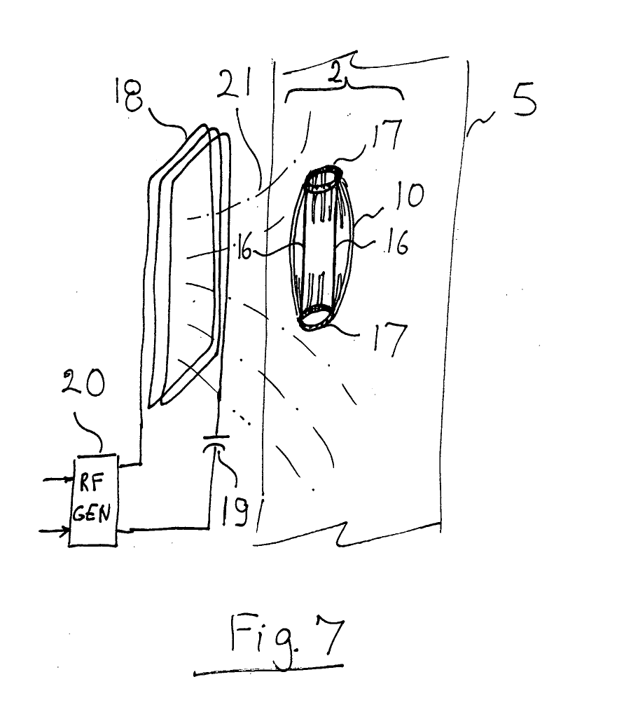

FIGS. 6A and 6B show a valve capable of being activated by an alternating magnetic field, using an external coil as shown in FIG. 7. Cage 10 is made of a polymeric material, such as carbon fiber or glass fiber strips but it has additional metallic rings 17 at both ends. The metallic rings 17 are joined by several Nitinol actuator wires 16. When placed in an alternating magnetic field a current is induced in the closed electrical path formed by wires 16 and metallic rings 17. The current heats up the Nitinol actuator wires causing them to contract, forcing the elastic cage 10 into a barrel shape, as shown in FIG. 6B. The expanding ribs pull elastic body 8 with then, opening tube 1 to the maximal flow position. Body 8 can be a liquid, a gel, an elastomer or a foam. A silicone rubber closed cell foam works well as it provides thermal insulation for wires 16, allowing them to heat up with minimal current and transfer minimal amount of heat to the blood. By the way of example, for a valve having a diameter of 6 mm and 20 mm long, two Nitinol wires of 0.25 mm diameter and 20 mm length were used, as shown in FIG. 6. Wires were of the Flexinol brand supplied by Dynalloy (www.dynalloy.com). The current needed to contract them by about 5% was about 0.7 A. The silicone rubber foam 8 was cut at the two places the wires are mounted to allow wires to move longitudinally and radially. Cage 10 was made of thin carbon fiber strips crimped into type 316L stainless rings 17. Body 8 can be bonded to tube 1 but valve will function properly even without this bonding, as blood pressure is pushing tube 1 towards body 8.

The external device needed to actuate the valve is shown in FIG. 7. A coil 18 is resonated with a series capacitor 19 and connected to a radio frequency (RF) generator 20. Alternating magnetic field 21 engages the loop formed by wires 16 and rings 18 and generates a heating current shortening the wires by about 5%. As long as the coil is powered, the valve 2 will stay open. For best coupling coil 8 is placed on the body of the patient above the location of the valve with the plane of the coil parallel to the plane defined by wires 16. By the way of example, a coil was made from 50 turns of 0.8 mm diameter enameled copper wire wound on a 6×6 cm frame. The inductance of the coil was about 150 uH. When resonated at 180 KHz with a capacitor 19 of 0.005 uF, the power coupling efficiency into the above described valve was about 2%. Since the power needed by the valve to stay open was about 0.5W the total power to the coil was about 25W. The RF generator was simply an NE555 timer set for 180 KHz and driving a MOSFET power switch. The fact the RF generator supplies a square wave rather than a sine wave is of no importance, since the high Q of the resonant circuit filters out all the harmonics and the current is sinusoidal.

While the disclosure uses an application for dialysis as an example, the invention can be used in all medical applications were a flow rate needs to be controlled while maintaining a smooth flow of fluids. Also, the words “round” and “round tube” should be broadly interpreted as any round, oval and rounded polygonal shape not having any sharp corners or transitions.

1. An implantable valve for controlling blood flow in the body by changing the diameter of a round tube and having a smooth transition between the different diameters inside said tube.

2. An arteriovenous shunt incorporating a valve controlling blood flow by changing the diameter of a flexible round tube, said tube having a smooth transition between the different diameters inside said tube.

3. A valve for controlling blood flow into an arteriovenous fistula, said valve controlling blood flow by changing the diameter of a flexible round tube, said tube having a smooth transition between the different diameters inside said tube.

4. A valve as in claim 1 wherein said diameter is changed by moving a part of said valve.

5. A valve as in claim 1 wherein said diameter is changed by moving a part of said valve by using a magnetic field.

6. A valve as in claim 1 wherein said diameter is changed by moving a part of said valve by using a Nitinol actuator wire heated by a changing magnetic field.

7. A valve as in claim 1 wherein said diameter is changed by adding or removing fluid from said valve.

8. A valve as in claim 1 wherein said diameter is changed by shortening a flexible cage causing it to expand radially as it is shortened, said expansion coupled to said tube.

9. A valve as in claim 2 wherein said diameter is changed by moving a part of said valve.

10. A valve as in claim 2 wherein said diameter is changed by moving a part of said valve by using a magnetic field.

11. A valve as in claim 2 wherein said diameter is changed by moving a part of said valve by using a Nitinol actuator wire heated by a changing magnetic field.

12. A valve as in claim 2 wherein said diameter is changed by adding or removing fluid from said valve.

13. A valve as in claim 2 wherein said diameter is changed by shortening a flexible cage causing it to expand radially as it is shortened, said expansion coupled to said tube.

14. A valve as in claim 3 wherein said diameter is changed by moving a part of said valve.

15. A valve as in claim 3 wherein said diameter is changed by moving a part of said valve by using a magnetic field.

16. A valve as in claim 3 wherein said diameter is changed by moving a part of said valve by using a Nitinol actuator wire heated by a changing magnetic field.

17. A valve as in claim 3 wherein said diameter is changed by adding or removing fluid from said valve.

18. A valve as in claim 3 wherein said diameter is changed by shortening a flexible cage causing it to expand radially as it is shortened, said expansion coupled to said tube.

19. A valve as in claim 1 wherein said tube is a blood vessel.

20. A valve as in claim 2 wherein said tube is a continuous part of said shunt.
